(19) United States
(12) Patent Application Publication
(10) Pub. No.: US 2025/0040893 A1
(43) Pub. Date: Feb. 6, 2025

(54) MEDICAL ASSEMBLIES, DEVICES, SYSTEMS, AND RELATED METHODS FOR MULTIPLE SENSORS

(71) Applicant: Boston Scientific Scimed, Inc., Maple Grove, MN (US)

(72) Inventor: Michael McGOVERN, San Diego, CA (US)

(73) Assignee: Boston Scientific Scimed, Inc., Maple Grove, MN (US)

(21) Appl. No.: 18/788,657

(22) Filed: Jul. 30, 2024

Related U.S. Application Data
(60) Provisional application No. 63/516,881, filed on Aug. 1, 2023.

(51) Int. Cl. A61B 5/00; A61B 1/00; A61B 5/0205; G16H 40/63

(52) U.S. Cl. CPC A61B 5/7225; A61B 1/00097; A61B 5/0205; G16H 40/63

(57) ABSTRACT

A medical system includes a processing unit, a medical device electrically coupled to the processing unit, and an electronic assembly disposed at a distal portion of the insertion portion. The medical device includes an insertion portion configured to be inserted into a body lumen of a subject. The electronic assembly includes at least a first sensor, a second sensor, and a switch. The electronic assembly is electrically coupled to the processing unit. The switch is configured to switch between the first sensor and the second sensor based on an instruction of the processing unit.

### CROSS-REFERENCE TO RELATED APPLICATIONS

This application claims the benefit of priority of U.S. Provisional Patent Application No. 63/516,881, filed on Aug. 1, 2023, the entirety of which is incorporated herein by reference.

### TECHNICAL FIELD

This disclosure generally relates to medical assemblies, devices, systems, and related methods for devices having a plurality of sensors. More particularly, at least some embodiments of the present disclosure relate to assemblies, devices, systems, and related methods for managing signals from a plurality of sensors of a distal tip of a medical device.

### BACKGROUND

Medical devices are often inserted into the body to perform a therapeutic and/or diagnostic procedure inside a subject's body. An example of such a device is an endoscope or other type of scope, which includes an insertion portion that is introduced into the body for diagnostic or therapeutic purposes. An insertion portion of an endoscope is inserted into the subject's body through an opening (e.g., a natural opening or an incision) and is delivered to a site inside the body, for example, through a body lumen. In one example, an endoscope may be inserted into a subject's mouth and advanced through the subject's esophagus.

Endoscopes or other scopes may include a variety of features, for example, to assist in performing a therapeutic and/or diagnostic procedure inside the subject's body. For example, a distal tip of an endoscope may include an imaging device that allows a surgeon to visualize the interior of the subject's body (e.g., a body lumen) from outside the body and remotely operate the endoscope to perform a desired diagnostic/therapeutic procedure. The distal tip may also include one or more illumination devices (e.g., LEDs, optical fibers, etc.), openings, elevators, or other features. Size constraints may limit the number or types of components that may be positioned at the distal tip of an endoscope. Therefore, medical assemblies, systems, and related methods are needed for incorporating components in a distal tip of an endoscope. The systems and methods of this disclosure may rectify some of the deficiencies described above or address other aspects of the art.

### SUMMARY

Examples of this disclosure relate to, among other things, systems, devices, and methods for performing one or more medical procedures with medical devices. Specifically, this disclosure includes medical systems and devices comprising at least two sensors and methods of use thereof (e.g., methods of switching between at least two sensors). Each of the examples disclosed herein may include one or more of the features described in connection with any of the other disclosed examples.

According to one aspect, a medical system may include a processing unit, a medical device electrically coupled to the processing unit, and an electronic assembly disposed at a distal portion of the insertion portion. The medical device may include an insertion portion configured to be inserted into a body lumen of a subject. The electronic assembly may include at least a first sensor, a second sensor, and a switch. The electronic assembly may be electrically coupled to the processing unit. The switch may be configured to switch between the first sensor and the second sensor based on an instruction of the processing unit.

In some configurations, the medical device may include a conducting element configured to transmit a composite waveform comprising signals from the first sensor and the second sensor. The composite waveform may be transmitted via a single conducting element. The conducting element may extend through a shaft of the medical device.

The processing unit may include a field programmable gate array (“FPGA”). The FPGA may be configured to generate the instruction to switch the switch between the first sensor and the second sensor. The instruction to switch between the first sensor and the second sensor may be transmitted from the FPGA to the electronic assembly via a cable electrically coupled to the FPGA and the electronic assembly.

In some configurations, the medical system may further include an output. The output may be electrically coupled to the FPGA. The FPGA may be configured to transmit data received from a composite waveform to the output.

The switch may be electrically coupled to an analog processing unit. The analog processing unit may be electrically coupled to an analog to digital converter. The analog to digital converter may be electrically coupled to an FPGA.

In some configurations, the electronic assembly may include a third sensor. and wherein the switch is configured to switch between the first sensor, the second sensor, and the third sensor based on the instruction received from the processing unit.

Each of the first sensor and the second sensor is an analog sensor. The first sensor and the second sensor may each be one of an image sensor, a temperature sensor, a humidity sensor, a light sensor, a flow rate sensor, a pressure sensor, an oximeter, a glucometer, a heart rate sensor, a respiration rate sensor, a force sensor, an airflow sensor, a position and/or orientation sensor, a magnetic field sensor, and a pH sensor.

In some configurations, the electronic assembly may include a first portion and a second portion. The first portion may include the first sensor and the switch. The second portion may include the second sensor. The first portion may be disposed on a first surface of the medical device. The second portion may be disposed on a second surface of the medical device. The first surface may be angled relative to the first surface.

According to another aspect, a medical method may include receiving data from a first sensor when a switch is in a first configuration, sending an instruction to move the switch from the first configuration to a second configuration, receiving data from the second sensor when the switch is in the second configuration, and transmitting the data received from the first sensor and the second sensor to a processing unit. The method may further include analyzing the transmitted data from the first sensor and the second sensor in order to reconstruct data from the first sensor and the second sensor. The transmitted data may be analyzed based on stored data of the instruction to move the switch. The method may further include sending an instruction to move the switch from the second configuration to a third configuration. In the third configuration, data may be received from a third sensor.

According to another aspect, an electronic assembly of a medical device may include a first sensor, a second sensor, a switch, and a conductive element. The switch may have a primary terminal. The switch may be configured to switch between a first terminal electrically coupled to the first sensor and a second terminal electrically coupled to the second sensor. The conductive element may be electrically coupled to the primary terminal. The conductive element may be configured to transmit a composite waveform comprising signals from the first sensor and the second sensor.

Any of the examples described herein may have any of these features in any combination.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 is a perspective view of a medical system according to aspects of this disclosure.

FIG. 2A illustrates a schematic, partial perspective view of a distal portion of a medical device of the medical system of FIG. 1, according to aspects of this disclosure.

FIG. 2B illustrates a schematic, partial perspective view of an alternative configuration of a distal portion of the medical device of the medical system of FIG. 1, according to aspects of this disclosure.

FIG. 2C illustrates a schematic, partial perspective view of an alternative configuration of a distal portion of the medical device of the medical system of FIG. 1, according to aspects of this disclosure.

FIG. 3 depicts a schematic diagram of a medical system, according to aspects of this disclosure.

FIG. 4 depicts an exemplary waveform of the electronic system of FIG. 3, according to aspects of this disclosure.

FIG. 5 depicts a flow diagram of an exemplary method, according to aspects of this disclosure.

FIG. 6 depicts a flow diagram of an alternative exemplary method, according to aspects of this disclosure.

FIG. 7 depicts an alternative exemplary waveform of the electronic system of FIG. 3, according to aspects of this disclosure.

FIG. 8A depicts an exemplary waveform of a first sampling scheme, according to aspects of this disclosure.

FIG. 8B depicts an exemplary waveform of a second sampling scheme, according to aspects of this disclosure.

### DETAILED DESCRIPTION

Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., patient). By contrast, the term “proximal” refers to a portion closest to the user when placing the device into the subject. Proximal and distal directions are labeled with arrows marked “P” and “D,” respectively, throughout various figures.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of +10% in a stated value or characteristic. Additionally, terms that indicate the geometric shape of a component/surface encompass both exact and approximate shapes.

Although a procedure site is discussed herein as being in a subject's gastrointestinal tract, this disclosure is not so limited, as the procedure site may be any internal lumen, organ, cavity, or other tissue within the subject. Additionally, although endoscopes are referenced herein, it will be appreciated that the disclosure encompasses various devices that may be inserted into a body of a subject, such as ureteroscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, or catheters.

Although medical assemblies, systems, and related methods are described herein, the disclosure shall not be so limited. For example, the assemblies, systems, and methods described herein may be applicable to any device having limited space to incorporate sensors.

Endoscopes may have limited space in which to incorporate components because the endoscope must have a diameter appropriate for insertion into a body lumen of a subject. For example, a distal tip of the endoscope may have a limited space in which to incorporate elements such as a working channel opening, an imaging device (e.g., camera), lighting elements (e.g., light emitting diodes (LEDs) or fiber optic elements). Distal tips may have limited space in which to incorporate additional features such as, for example, sensors (e.g., image, temperature, pressure, position, magnetic field, light, force, humidity, or other type(s) of sensors). The sensors may be utilized, for example, to provide data associated with a therapeutic or diagnostic procedure performed inside the subject's body. Incorporating multiple sensors may pose various challenges. For example, each sensor may require additional cabling, which may increase the cost and/or complexity of the endoscope and its assembly. Sensors including circuitry for converting analog waveforms into digital signals may be relatively large, posing challenges for fitting multiple sensors having such circuitry within a distal tip of an endoscope. Embodiments of the disclosure may address one or more of the limitations in the art. The scope of the disclosure, however, is defined by the attached claims and not the ability to solve a specific problem.

The disclosure is drawn to assemblies, devices, systems, and related methods, for switching between multiple sensors, among other aspects. In an aspect, a system may include an electronic assembly of a distal tip of an endoscope, as well as processing circuitry at a handle of the endoscope and/or in an external controller. In aspects, a distal tip of the endoscope may include a plurality of sensors. An electronic assembly for the distal tip of the endoscope may include a chip or other type of circuit board having a plurality of analog sensors positioned thereon. The electronic assembly may further include a switching mechanism. The switching mechanism may combine signals from all or a subset of the plurality of sensors into a single, composite waveform. A single cable may carry the composite waveform, decreasing a number of cables required from one cable for each sensor to one cable for a plurality of sensors. Processing circuitry may be disposed in a handle of the endoscope and/or in external controller. The processing circuitry may include, for example, an analog processing unit, an analog to digital converter, a field programmable gate array, and/or a microcontroller. The processing circuitry may be operable to control the switching mechanism and/or to process a signal received from the electronic assembly of the distal tip of the endoscope. For example, the processing circuitry may operate the switching mechanism so as to receive a signal from a particular sensor at a given time. The processing circuitry also may reconstruct data from each of the plurality of sensors.

This disclosure is described with reference to exemplary medical systems for accessing a target site of a subject, for example, to measure one or more aspects of the target site by switching between multiple sensors on the endoscope. This may provide improved medical tool functionality and/or assist medical professionals with performing medical procedures. However, it should be noted that reference to any particular device and/or any particular procedure is provided only for convenience and not intended to limit the disclosure. A person of ordinary skill in the art would recognize that the concepts underlying the disclosed devices and application methods may be utilized in any suitable procedure, medical or otherwise. The assemblies and systems described herein may be used in conjunction with other types of medical devices. This disclosure may be understood with reference to the following description and the appended drawings, wherein like elements are referred to with the same reference numerals.

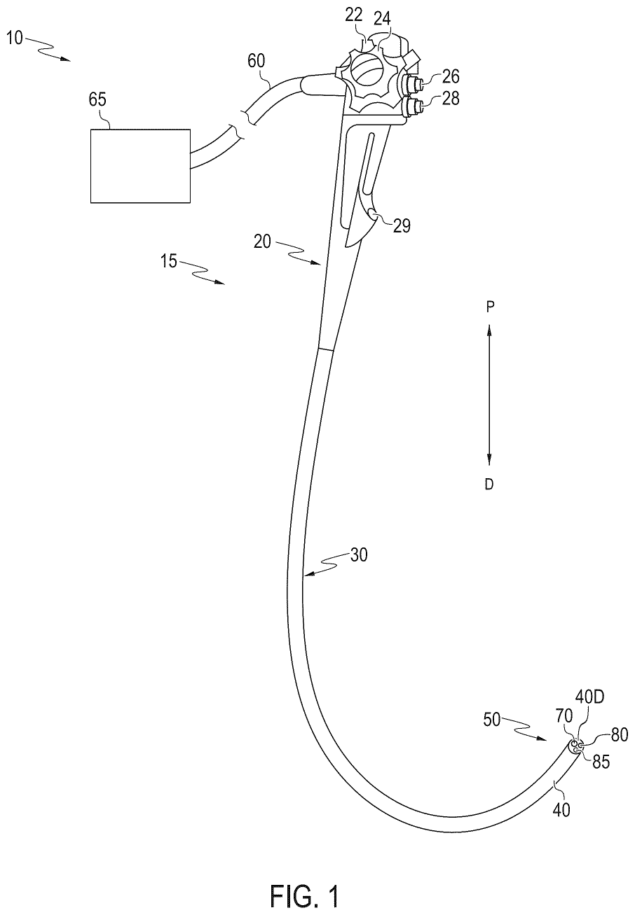

Referring to FIG. 1, a medical system 10 according to an exemplary embodiment is shown. Medical system 10 may include a medical device 15 comprising a handle 20 and an insertion portion 30 (e.g., a shaft or a catheter). Insertion portion 30 may be connected to a distal portion of handle 20. Insertion portion 30 may terminate distally in a distal tip 50. An umbilicus 60 extends from a proximal portion of handle 20.

Umbilicus 60 may be removably coupled (e.g., directly or indirectly) to a processing unit 65. Processing unit 65 may be configured to process information (e.g., sensor data, imaging data, light data, etc.) received from medical device 15. In some aspects, processing unit 65 may be a controller or capital equipment associated with medical device 15. Umbilicus 60 may include one or more electrical cables and/or light cables for coupling to processing unit 65 via, e.g., a removable connector. Although not shown, processing unit 65 may include a visual output (e.g., an internal monitor or screen) or processing unit 65 may be coupled to a visual output (e.g., an external or separate monitor or screen). Although not shown, umbilicus 60 may additionally or alternatively include one or more lumens for supplying gas or liquid to handle 20 and/or insertion portion 30.

Handle 20 may include a first actuator 22 and/or a second actuator 24. First actuator 22 and/or second actuator 24 may include, for example, rotatable knobs that rotate to push/pull one or more elements that extend through insertion portion 30 and connect to a distal end portion 40 of insertion portion 30. For example, first actuator 22 and/or second actuator 24 may be configured to rotate about their axes to push/pull actuating elements (e.g., wires, not shown) which extend within one or more lumens of insertion portion 30. Rotation of first actuator 22 and/or second actuator 24 may cause insertion portion 30 and/or distal end portion 40 to bend, for example, via an articulating joint (not shown). Additionally or alternatively, handle 20 may include additional actuators (e.g., buttons, knobs, levers, locks, etc.) to, for example, limit movement of the first actuator 22 and/or second actuator 24, close or open a forceps, rotate an end effector about a longitudinal axis, raise or lower an elevator, capture an image, and/or provide other functionality to an end effector and/or distal end portion 40.

Handle 20 may also include a first valve 26 and a second valve 28. Although two valves (i.e., first valve 26 and second valve 28) are shown, handle 20 may include additional (e.g., a third valve, a fourth valve, etc.) or fewer valves (e.g., no valves or first valve 26). In some embodiments, first valve 26 may be configured to control the supply of air and/or water to distal end portion 40. Second valve 28 may be configured to control the application of suction to distal end portion 40. Additional valves may be used, for example, to control the application of one or more medicines, agents, materials, etc. to distal end portion 40.

Handle 20 may also include a proximal opening 29, which may be fluidly connected to one or more lumens of insertion portion 30. For example, a medical instrument (not shown) may be inserted into proximal opening 29 and may be extended to distal end portion 40 via the one or more lumens. The lumen(s) may have a distal opening 85 at distal tip 50, which may be a distalmost end of distal end portion 40, which may be fluidly coupled to proximal opening 29. In such a way, a medical instrument inserted into proximal opening 29 may be extended distally through distal opening 85 and extend distally from distal opening 85. Additionally or alternatively, one or more materials (e.g., liquids, gels, gasses, patches, powders, etc.) may be supplied to a target site via distal opening 85. Additionally or alternatively, suction may be supplied via proximal opening 29, for example, to remove debris from the target site via distal opening 85.

Insertion portion 30 may be flexible and may be formed of any medical grade material suitable for accessing a tortuous path within the body. The rigidity/flexibility of insertion portion 30 is not limited. In some embodiments, the rigidity/flexibility of insertion portion 30 may vary, for example, along a longitudinal length of insertion portion 30. A longitudinal length of insertion portion 30 may vary and is not limited. For example, insertion portion 30 may have a length of approximately 5-100 inches, for example, between 10-70 inches. Furthermore, an outer diameter of insertion portion 30 is not limited. For example, the outer diameter of insertion portion 30 may be approximately 0.07-0.60 inches, for example, between 0.10-0.50 inches. In some embodiments, the outer diameter of insertion portion 30 may vary along the longitudinal length. For example, the outer diameter of a proximal portion of insertion portion 30 may be less than or greater than the outer diameter of a distal portion of insertion portion 30 (e.g., distal portion 40), or vice versa.

Although not shown, distal end portion 40 may include one or more articulation joints. Distal tip 50 may include one or more end effectors, and/or openings for suction, irrigation, insufflation, accessory devices, etc. Distal end portion 40 may further include one or more devices 70. Device(s) 70 may include one or more visualization devices (e.g., cameras, image sensors, lenses, etc.), one or more illumination devices (e.g., LEDs, optical fibers, etc.), one or more treatment devices (e.g., laser fibers, elevators, etc.), and/or one or more other devices to otherwise image, view, or otherwise treat a treatment site. For example, device(s) 70 may include one or more charge-coupled device(s) (CCD) and/or one or more complementary metal-oxide semiconductor (CMOS) imaging sensors. Device(s) 70 may be electrically coupled (e.g., directly or indirectly) to processing unit 65, for example, via wires and/or cables extending through insertion portion 30, through handle 20, and through umbilicus 60.

Medical device 15 may also include at least one sensor array 80. Sensor array 80 may be electrically coupled (e.g., directly or indirectly) to processing unit 65. For example, at least one cable and/or wire may extend proximally from sensor array 80, through insertion portion 30, handle 20, and/or umbilicus 60, to processing unit 65. Although not shown in FIG. 1, the at least one cable or wire extending proximally from sensor array 80 may extend through one or more lumens (not shown) of insertion portion 30. In other examples, the at least one cable or wire is at least partially embedded within material of insertion port 30. Alternatively, the at least one cable or wire of sensor array 80 may extend externally to insertion portion 30, for example, along an external surface of insertion portion 30. The at least one cable or wire of sensor array 80 may be shielded, for example, to help limit or reduce signal noise. The at least one cable or wire of sensor array 80 and/or respective terminations of the at least one cable or wire may be impedance controlled. The shielding and/or impedance control may help to ensure that the signal(s) transmitted via the at least one cable or wire are not affected by noise or otherwise negatively affected by surroundings.

Sensor array 80 may be coupled to one or more portions of distal tip 50. For example, sensor array 80 may be coupled to a distal face 40D of distal tip 50 of insertion portion 30. In some embodiments, sensor array 80 may additionally or alternatively be coupled to one or more portions of insertion portion 30, for example, proximal or adjacent to distal face 40D and/or distal tip 50.

Although FIG. 1 depicts medical device 15 as being a forward-facing device (e.g., with distal opening 85, device(s) 70, and sensor array 80 as facing distally, approximately parallel to a longitudinal axis of insertion portion 80), medical device 15 may alternatively be a side-facing device. In other words, one or more of distal opening 85, device(s) 70, and/or sensor array 80 may face radially outward (e.g., approximately perpendicularly to a longitudinal axis of insertion portion 30). Alternatively, medical device 15 may have both forward-facing and side-facing elements.

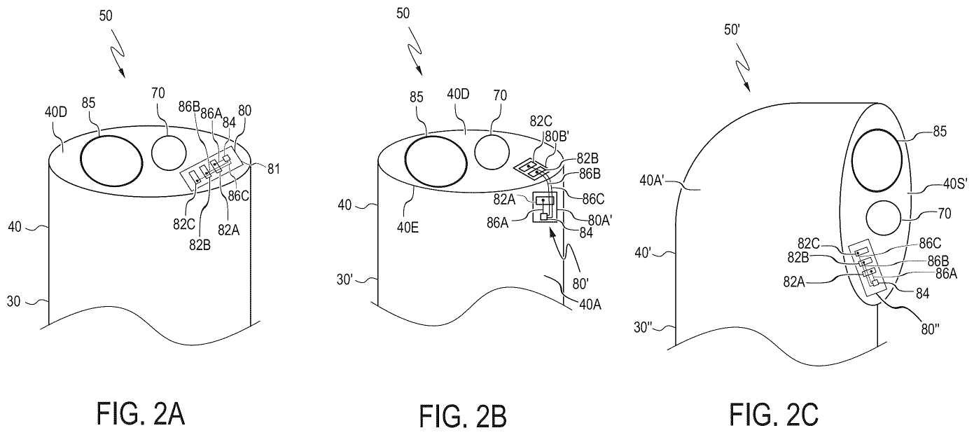

FIG. 2A illustrates an exemplary configuration of sensor array 80 oriented on distal face 40D of distal tip 50 of distal portion 40 of insertion portion 30. It will be appreciated that FIG. 2A is merely a schematic representation, and the elements of distal tip 50/distal face 40D may have any suitable arrangement or shape. As shown in FIG. 2A, distal portion 40 may be a distal portion of a forward-facing scope such as, for example, an endoscope, a gastroscope, a colonoscope, a bronchoscope, a ureteroscopes, a cholangioscope, or any other scope or medical device having a distal face such as distal face 40D, with components thereof facing approximately distally (i.e., approximately parallel to a longitudinal axis of insertion portion 30). Distal face 40D may include device(s) 70 and/or distal opening(s) 85, as previously discussed above with respect to FIG. 1. Although one device 10, distal opening 85, and sensor array 80 are depicted on distal face 40D, it will be appreciated that distal face 40D may include any suitable number of such elements. Additionally or alternatively, an interior of device 50 may include one or more device(s) 70 and/or sensor array(s) 80. For example, sensor array 80 may be oriented adjacent to device(s) 70 and/or distal opening(s) 85 on distal face 40D. Sensor array 80 may be fixed to distal surface 40D by, for example, one or more adhesives, fasteners, or any method commonly used in the art to permanently or temporarily fix sensor array 80 to distal surface 40D. Alternatively, portions of sensor array 80 may be disposed within a housing of distal tip 50.

In some embodiments, sensor array 80 may be partially or completely embedded into or encapsulated by a material (e.g., a plastic, an acrylic, a metal, a polymer, etc.) forming at least a portion of distal surface 40D. For example, sensor array 80 may be molded into distal surface 40D. Alternatively, sensor array 80 may be disposed at least partially within distal tip 50, and encapsulation material may be utilized to secure sensor array 80 within distal tip 50. Although not shown, at least one wire or cable may extend proximally from sensor array 80, for example, through distal tip 50 and an internal lumen (not shown) of insertion portion 30. Additionally or alternatively, the at least one wire or cable may extend from sensor array 80 through distal opening(s) 85 and into the lumen in communication with proximal opening 29 . . . .

Sensor array 80 may include a substrate 81, such as a rigid circuit board, a flexible circuit board, or a circuit board having rigid and flexible portions. For example, substrate 81 may include a rigid printed circuit board (PCB) and/or a flexible PCB. In examples, substrate 81 may be comprised of rigid and/or flexible materials integrated into one circuit. A shape and/or a size of sensor array 80/substrate 81 may vary. For example, sensor array 80/substrate 81 may be square, circular, ovular, triangular, polygonal, or any other shape.

Sensor array 80 may include a first sensor 82A, a second sensor 82B, a third sensor 82C, and a switching mechanism 84. Although three sensors (i.e., first sensor 82A, second sensor 82B, and third sensor 82C) are shown and described herein, sensor array 80 may include fewer or additional sensors. For example, sensor array 80 may include two sensors, four sensors, five sensors, etc. In some examples, sensor array 80 may include a plurality of switching mechanisms 84 and a plurality of sensors 82A, 82B, 82C, etc. associated with each of the plurality of switching mechanisms 84. Each sensor may be an analog sensor, such as a sensor that is configured to produce a continuous analog output signal. Sensor array 80 may also include other elements (e.g., digital sensors, or any other type of electronic element).

Each sensor of sensor array 80 may be electrically coupled to switching mechanism 84 via one or more electrical connections. The electrical connections may include trace(s) on substrate 81, waveguide(s) on substrate 81, wire(s) and/or cable(s). For example, first sensor 82A may be electrically coupled (e.g., directly or indirectly) to switching mechanism 84 by a first electrical connection 86A; second sensor 82B may be electrically coupled (e.g., directly or indirectly) to switching mechanism 84 by a second electrical connection 86B; and third sensor 82C may be electrically coupled (e.g., directly or indirectly) to switching mechanism 84 by a third electrical connection 86C. Additional electrical connection(s) may be utilized to couple additional sensors to switching mechanism 84.

Each sensor (i.e., first sensor 82A, second sensor 82B, third sensor 82C, etc.) may be configured to measure, detect, indicate, and/or respond to one or more aspects of the subject and/or a device such as, for example, medical device 15 and/or an accessory or auxiliary device (not shown). Accordingly, a variety of sensors may be used. For example, the sensors (i.e., first sensor 82A, second sensor 82B, third sensor 82C, etc.) may include one or more of an image sensor, a temperature sensor, a humidity sensor, a light sensor, a flow rate sensor, a pressure sensor, an oximeter, a glucometer, a heart rate sensor, a respiration rate sensor, a force sensor, an airflow sensor, a position and/or orientation sensor, a magnetic field sensor, a pH sensor, or any other type of sensor.

In some embodiments, each sensor (i.e., first sensor 82A, second sensor 82B, third sensor 83C, etc.) may be a same type of sensor. For example, each of first sensor 82A, second sensor 82, third sensor 83C, etc. may be a temperature sensor. Alternatively, each sensor (i.e., first sensor 82A, second sensor 82B, third sensor 83C, etc.) may be a different type of sensor. For example, first sensor 82A may be an image sensor, second sensor 82B may be a temperature sensor, third sensor 82C may be a humidity sensor, etc. In further alternative embodiments, some sensors may be the same type of sensor, and other sensors may be different types of sensors. For example, first sensor 82A and second sensor 82B may each be temperature sensors, third sensor 82C may be a light sensor, etc. The above examples are merely illustrative, and any combination of sensors may be utilized within the scope of the disclosure.

Switching mechanism 84 may be operable to switch among sensors 82A, 82B, 82C, etc. For example, at a given time, switching mechanism 84 may selectively electrically couple one of sensors 82A, 82B, 82C, etc. to wires/cables extending proximally from distal tip 50, through insertion portion 30. As discussed in detail below, processing unit 65 may control switching mechanism 84 to switch among sensors 82A, 82B, 82C, so as to select from which of sensors 82A, 82B, 82C to transmit a signal.

FIG. 2B illustrates an alternative exemplary configuration of a sensor array 80′ oriented on distal portion 40 of the front-viewing scope previously described with respect to FIG. 2A. Sensor array 80′ may include any and all of the characteristics described above with respect to sensor array 80 of FIG. 2A. In this configuration, sensor array 80′ may include a first portion 80A′ coupled to distal face 40D (or within distal tip 50 and facing distally) and a second portion 80B′ coupled to an outer surface 40A (e.g., a radially outer surface) of distal tip 50, for example, proximal to distal face 40D (or within distal tip 50 and facing radially outward). In such a way, distal face 40D and outer surface 40A of distal tip may be angled relative to one another such that first portion 80A′ and second portion 80B′ are angled relative to one another. For example, distal face 40D may be angled approximately 20-90 degrees relative to outer surface 40A.

As shown in FIG. 2B, sensor array 80′ may include two substrates 81A′ (portion 80A′), 81B′ (portion 80B′). Alternatively, sensor array 80′ may include a single substrate that is flexible or partially flexible, such that the substrate may be bent to form first portion 80A′ and/or second portion 80B′.

As shown in FIG. 2B, first portion 80A′ may include first sensor 82A and switching mechanism 84, and second portion 80B′ may include second sensor 82B and third sensor 82C. However, such an arrangement is merely exemplary, and any suitable arrangement may be utilized. First electrical connection 86A may extend from first sensor 82A on first portion 80A′ to switching mechanism 84. Each of second electrical connection 86B and third electrical connection 86C may extend from second sensor 82B and third sensor 82C, respectively, on second portion 80B′ to switching mechanism 84 on first portion 80A′. For example, each of second electrical connection 86B and third electrical connection 86C may be bent or curved over an edge 40E formed between distal face 40D and outer surface 40A of distal portion 40. Second electrical connection 86B and/or third electrical connection 86C may be embedded into or partially encapsulated by a material forming distal face 40D and/or outer wall 40A of distal portion 40. Alternatively, second electrical connection 86B and/or third electrical connection 86C may extend from second portion 80B′, for example, along distal face 40D and/or outer wall 40A, to first portion 80A′.

Although two portions (i.e., first portion 80A′ and second portion 80B′) of sensor array 80′ are shown in FIG. 2B, sensor array 80′ may include additional portions/substrates (e.g., three portions/substrates, four portions/substrates, etc.). Each portion may include one or more additional sensors. Although not shown, additional sensor(s) may each be electrically coupled to switch mechanism 84 on first portion 80A′ by additional cables. Alternatively, sensor array 80′ may include a plurality of switches, coupled to various of the sensor(s). Each portion may be positioned on outer surface 40A and/or distal face 40D. For example, a third portion (not shown) may be coupled to outer surface 40A, proximal to first portion 80A′ or on distal face 40D, for example, adjacent to second portion 80B′. Additionally or alternatively, additional portions of sensor array 80′ may be positioned within opening(s) 85, for example, proximal to distal face 40D of distal portion 40, or otherwise within distal tip 50.

FIG. 2C illustrates a further alternative exemplary configuration of sensor array 80″ oriented on an alternative distal portion 40′ of insertion portion 30′. Sensor array 80″ may include any and all of the characteristics described above with respect to sensor array 80 of FIG. 2A and/or sensor array 80′ of FIG. 2B, except as described below. Additionally, distal portion 40′ may include any and all of the characteristics of distal portion 40, described above with respect to FIGS. 1, 2A, and 2B, except as described below. In this embodiment, distal portion 40′ includes a side face 40S′. In such an embodiment, a distal tip 50′/distal portion 40′ may be a distal portion of a duodenoscope or a side-viewing endoscope. Sensor array 80″ may be coupled to side face 40S′ of distal tip 50′. Opening(s) 85 and device(s) 70 may likewise be positioned on side face 40S′. Distal tip 50′/distal portion 40′ may be side facing; i.e., elements of distal tip 50′ may face radially outward (approximately perpendicularly to a longitudinal axis of insertion portion 30′). Side face 40S′ may have a plane that is approximately parallel to a longitudinal axis of insertion portion 30′.

Although not shown, sensor array 80″ may include two or more portions. The two or more portions may be similar to first portion 80A′ and second portion 80B′ of sensor array 80′, discussed above with respect to FIG. 2B. The two or more portions of sensor array 80″ may be positioned on various portions of distal portion 40′. For example, although not shown, sensor array 80″ may include a first portion positioned on side face 40S′, a second portion may be positioned on a distalmost end of distal tip 50′ of distal portion 40′, and/or a third portion may be positioned on an outer surface 40A′ of distal tip 50′. Additional portions of sensor array 80″ may additionally or alternatively be positioned within opening(s) 85, for example, within a lumen of insertion portion 30, or otherwise within distal tip 50′.

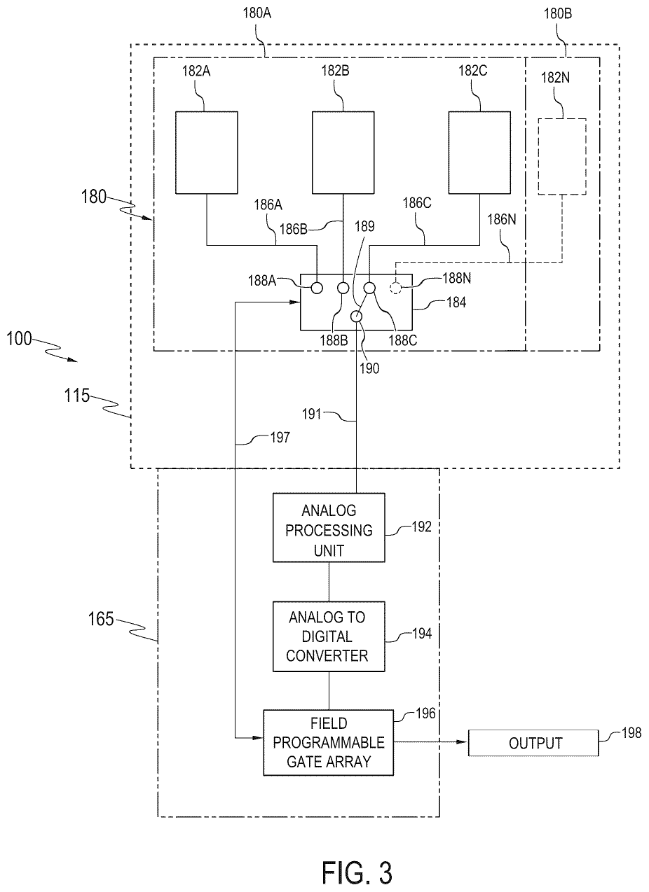

FIG. 3 illustrates a schematic diagram of electronic elements of an exemplary medical system 100, including a medical device 115, and a processing unit 165. Medical system 100, including medical device 115 and processing unit 165, may have any or all of the characteristics of medical system 10. For example, medical device 115 may have any of the properties of medical device 15, and processing unit 165 may have any of the properties of processing unit 65, described above with respect to FIG. 1. Although not separately labeled in FIG. 3, medical device 115 may include handle 20, insertion portion 30, and/or umbilicus 60 of FIG. 1. Medical device 115 may include at least one sensor array 180. Sensor array 180 may have any or all of the characteristics of sensor array 80 of FIGS. 1 and 2A, sensor array 80′ of FIG. 2B, and/or sensor array 80″ of FIG. 2C. For example, sensor array 180 may include a first sensor 182A, a second sensor 182B, and a third sensor 182C. Sensor array 180 may further include one or more additional sensor(s) 182N. As discussed above, a number of sensors depicted in FIG. 3 is merely exemplary, and any suitable number of sensors may be utilized.

Sensor array 180 may also include a switching mechanism 184. In some embodiments, sensor array may include a first portion 180A and a second portion 180B. For example, first portion 180A of sensor array 180 may include first sensor 182A, second sensor 182B, third sensor 182C, and switching mechanism 184. First portion 180A may alternatively include fewer or additional sensors and/or other elements. Second portion 180B may include additional sensor(s) 182N. For example, second portion 180B may include a fourth sensor, a fifth sensor, etc. In alternatives, switching mechanism 184 may be disposed on second portion 180B, with or without sensor(s) 182N.

As previously described, each sensor (e.g., first sensor 182A, second sensor 182B, third sensor 182C, etc.) may be coupled to switching mechanism 184 by one or more electrical connections (e.g., trace(s), waveguide(s), wire(s), and/or cable(s)). For example, as shown in FIG. 3, first sensor 182A is coupled to a first terminal 188A of switching mechanism 184 by a first electrical connection 186A, second sensor 182B is coupled to a second terminal 188B of switching mechanism 184 by a second electrical connection 186B, third sensor is coupled to a third terminal 188C of switching mechanism 184 by a third electrical connection 186C, etc. Sensor array 180 may include an equal number of sensors 182A, 182B, 182C, etc. and terminals 188A, 188B, 188C, etc. Each additional sensor 182N may have a terminal 188N. For each additional terminal 188N, an electrical connection 186N may extend between terminal 188N and sensor 182N.

A switch 189 of switching mechanism 184 may be configured to switch among the terminals (i.e., first terminal 188A, second terminal 188B, third terminal 188C, etc.). In such a way, switching mechanism 184 may be a single pole, double throw switch type for embodiments with two sensors (e.g., first sensor 182A and second sensor 182B), a single pole, triple throw switch type for embodiments with three sensors (e.g., first sensor 182A, second sensor 182B, and third sensor 182C), or any suitable type of switch for a given number of sensors. Switch 189 may be configured to be movable to between each terminal (e.g., first terminal 188A, second terminal 188B, third terminal 188C, etc.) so as to complete a circuit between each terminal (e.g., first terminal 188A, second terminal 188B, third terminal 188C, etc.) and a primary terminal 190 of switch 189.

Medical system 100 may further include processing unit 165. Processing unit 165 may be a controller (e.g., a standalone controller, a mobile device, a tablet, etc.). Alternatively, processing unit 165 may be integrated with a handle of a medical device (e.g., handle 20 of medical device 15 shown in FIG. 1). Although various components are described as being disposed within processing unit 165, it will be appreciated that the components may be located within separate structures/housings of one or more portions of medical system 100. For example, some components may be disposed within an external controller, and other components may be disposed within a handle of a medical device, such as, for example, handle 20 of medical device 15 (shown in FIG. 1).

A cable or wire 191 (e.g., a conducting element) may extend between primary terminal 190 of switch 189 and processing unit 165. In such a way, wire 191 may extend directly between primary terminal 190 of switch 189 on sensor array 180 to processing unit 165. Although not shown, intervening structures may exist, for example, between primary terminal 190 and processing unit 165. In such a way, wire 191 may be extend through or be coupled to the intervening structure(s) such that an electrical connection is made between sensor array 180 and processing unit 165. For example, wire 191 may extend through a shaft of medical device 115 (e.g., through a shaft of insertion portion 30 of FIG. 1) to a connector of an umbilicus (e.g. umbilicus 60 of FIG. 1), where it may be selectively electrically connected to conductive elements (e.g., traces, waveguides, wires, or cables) of processing unit 165. Either directly or indirectly (e.g., via a connector of an umbilicus and/or circuit boards in a handle of medical device 115 (e.g., handle 20 of FIG. 1), wire 191 may extend proximally from sensor array 180 to an element of processing unit 165, such as analog processing unit. As mentioned above, wire 191 may be shielded and/or impedance controlled.

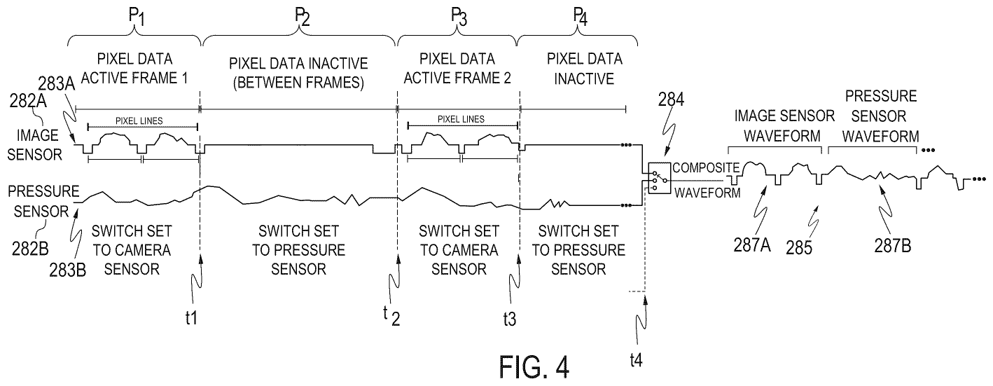

Accordingly, in this configuration, a total amount of wiring or cabling is reduced. For example, instead of a wire extending from each sensor, through medical device 115 (e.g., through insertion portion 30, handle 20, and/or umbilicus 60 shown in FIG. 1) to processing unit 165, wire 191 may be a single wire used for multiple sensors. Wire 191 may be configured to transmit a signal from each sensor (e.g., first sensor 182A, second sensor 182B, third sensor 182C, etc.). For example, depending on which sensor (e.g., first sensor 182A, second sensor 182B, third sensor 182C, etc.) switch 189 is electrically coupled to, a different signal may be transmitted through wire 191. A signal transmitted through wire 191 will be further discussed with respect to FIG. 4. Wire 191 may transmit the signal from the sensors directly or indirectly to processing unit 165.

Processing unit 165 may include analog processing unit 192, an analog to digital converter (ADC) 194, and a field programmable gate array (FPGA) 196, among other elements, such as, for example, a microcontroller. Although analog processing unit 192, ADC 194, and FPGA 196 are described as being elements of processing unit 165, these elements may be disposed in alternative structures (e.g., handle 20, a connector at the distal end of umbilicus 60 extending from handle 20, etc.). Analog processing unit 192, ADC 194, and FPGA 196 may be disposed within a single housing or within different elements/housings. In some examples, analog processing unit 192, ADC 194, and FPGA 196 may be disposed on a single substrate/chip. It will be appreciated that, in examples, one or more of analog processing unit 192, ADC 194, or FPGA may be omitted.

In some examples, wire 191 may be coupled to analog processing unit 192. Analog processing unit 192 may include one or more capacitors, resistors, indictors, transistors, op amps, comparators, or any other processing element or related circuitry known in the art to perform analog signal processing on received data. Analog processing unit 192 may be used to filter, amplify, and/or otherwise alter a signal received from the switching mechanism 184 via wire 191. For example, each of first sensor 182A, second sensor 182B, third sensor 182C, etc. may transmit data to analog processing unit 192 when switch 189 connects the respective terminal (i.e., first terminal, second terminal, third terminal, etc.) to primary terminal 190 of switching mechanism 184.

Analog processing unit(s) 192 may be electrically coupled to ADC 194 (e.g., via a wire, cable, trace, or waveguide). ADC 194 may be used, for example, to convert an analog signal received from analog processing unit(s) 192 to a digital signal. Any type of ADC known in the art may be utilized. For example, ADC 194 may be a single-ended ADC (e.g., an ADC with one input terminal) or a differential input (e.g., a high-speed ADC with two input terminals).

ADC 194 may be electrically coupled to FPGA 196 (e.g., via one or more wires, cables, traces, conductors, or waveguides). The digital signal from ADC 194 may thus be transmitted to FPGA 196. FPGA 196 may be configured to receive, sort, store, and/or reconstruct the digital signal received from ADC 194. For example, as described in detail below, FPGA 196 may be configured to reconstruct, sort, or otherwise analyze the composite signal transmitted from sensors 182A, 182B, 182C, etc. In some embodiments, the data that has been received, sorted, stored, and/or reconstructed by FPGA 196 may be transmitted from FPGA 196 to an output 198. Output 198 may include for example, a display or a monitor, one or more image processing units, a demosaic, and/or other blocks of an image processing pipeline. Further, FPGA 196 may also be configured to control switching mechanism 184, as described below, for example, via at least one cable or wire 197 (or another type of conducting element). For example, wire 197 may be configured to transmit an electrical signal from FPGA 196 to change switch 189 from terminal to terminal (e.g., from first terminal 188A to second terminal 188B, from second terminal 188B to third terminal 188C, etc.). The electrical signal may be an analog or digital signal.

In such a way, switch 189 switches among the various sensors (e.g., sensors 182A, 182B, 182C . . . 182N) upon receiving instructions from processing unit 165. For example, in a first position, switch 189 may form an electrical connection between first sensor 182A and analog processing unit 192 or another component of processing unit 165. Instructions may be sent from processing unit 165 (e.g., FPGA 196) to switching mechanism 184 to move switch 189 to a second position. In such a way, switch 189 moves from the first position to the second position based on the instruction received from processing unit 165. In the second position, switch 189 may form an electrical connection between second sensor 182B and analog processing unit 192 or another component of processing unit 165. Additional instructions (e.g., a second instruction) may be sent to switching mechanism 184 to move switch 189 to a third position such that switching mechanism is in a third configuration. In the third configuration, switch 189 may form an electrical connection between third sensor 182C and analog processing unit 192 or another component of processing unit 165. Additional configurations may be used to accommodate additional sensors. Additional instructions (e.g., a third instruction, a fourth instruction, etc.) may be sent to switching mechanism 184 to move switch 189 among the additional sensors.

Because switch 189 moves among the sensors (e.g., sensors 182A, 182B, 182C . . . 182N), a composite waveform is produced (shown in FIG. 4) and transmitted via wire 191. For example, when switch 189 is in the first configuration described above (connected to first sensor 182A), a first waveform from first sensor 182A may be transmitted along wire 191. In the second configuration of switch 189 (described above), a second waveform created by second sensor 182B may be transmitted along wire 191. Thus, if switch 189 switches from first sensor 182A to second sensor 182B, the second waveform may be appended (i.e., connected) to the first waveform and wire 191 may carry a composite waveform comprised of the first waveform and the second waveform. In such a way, the appended second waveform may not overlap or overwrite with the first waveform. In the third configuration (described above), a third waveform created by third sensor 182C may be transmitted along wire 191. If switch 189 switches from first sensor 182A to second sensor 182B and then to third sensor 182C, this third waveform may be appended to the second waveform and the first waveform. The above examples are merely exemplary. Any pattern or sequence of switching switch 189 may be utilized to generate composite waveforms from various of sensors 182A, 182B, 182C . . . 182N. A waveform transmitted by wire 191 may include a waveform that includes signals from first sensor 182A, second sensor 182B, third sensor 182C, etc. Thus, as switch 189 switches between terminals of various sensors, a composite waveform is created containing data from the selected sensors. Switch 189 and the various sensors may have electrical connections or interfaces that are impedance matched, for example, to help preserve signal quality.

Still referring to FIG. 3, the composite waveform may then be transmitted via wire 191 to analog processing unit 192, where the waveform may be filtered, amplified, and/or level shifted, as discussed above. In such a way, noise may be reduced from the composite waveform (e.g., a signal-to-noise ratio may be improved). The modified composite waveform may then be transmitted to ADC 194, where the waveform may be converted from the analog waveform to a digital signal. The digital signal may then be transmitted from ADC 194 to FPGA 196. The digital signal may be stored or sorted by FPGA 196, for example, to reconstruct the separate waveforms for each sensor. For example, FPGA 196 or a memory associated therewith may store information regarding how switch 189 was modulated (e.g., at which time and to which sensor 182A, 182B, 182C . . . 182N switch 189 was modulated). Alternatively, FPGA 196 or another component of processing unit 165 may analyze characteristics of the signal to identify known characteristics of signals from the sensor(s) 182A, 182B, 182C . . . 182N. In some examples, signals characteristics may include downwards square wave, or a voltage intensity baseline. FPGA 196 may be electrically coupled to an output (e.g., a monitor, a screen, a second processor, etc.) 198, either directly or indirectly (e.g., via other processing components). Information from FPGA 196 regarding first sensor 182A, second sensor 182B, third sensor 182C, etc. may then be displayed on output 198, stored, or undergo additional processing.

FIG. 4 shows how signals from two exemplary sensors may be combined in a single waveform. FIG. 4 shows data from two exemplary sensors of any of the above-mentioned sensor arrays (e.g., sensor array 80 of FIGS. 1 and 2A, sensor array 80′ of FIG. 2B, sensor array 80″ of FIG. 2C, or sensor array 180 of FIG. 3). FIG. 4 depicts data from an image sensor 282A and a pressure sensor 282B for illustrative purposes only. As previously described, alternative types of sensors may be used. Image sensor 282A may generate an exemplary image sensor analog signal/waveform 283A. Pressure sensor 282B may generate an exemplary analog pressure sensor signal/waveform 283B. As discussed in further detail below, analog waveforms 283A, 283B may be combined into a composite signal/waveform 285 via a switching mechanism 284 (having any of the properties of switching mechanisms 84, 184). For ease of illustration, dashed vertical lines depict first time t₁, second time t₂, third time t₃, and fourth time t₄. Before first time t₁ is a first time period P₁. Between first time t₁ and second time t₂ is a second time period P₂. Between second time t₂ and third time t₃ is a third time period P₃. Between third time t₃ and fourth time t₄ is a fourth time period P₄. Waveforms 283A and 283B are referenced at periods P₁, P₂, P₃, and P₄, for ease of illustration.

Image sensor 282A may be any suitable type of image sensor, such as a complementary metal oxide semiconductor (CMOS) sensor or a charge coupled device (CCD) sensor. Such examples are not limiting, and any type of image sensor may be utilized. As shown in FIG. 4, during first period P₁, pixel data from image sensor 282A may be transmitted from image sensor 282A. Such pixel data may be in the form of an analog image sensor waveform 283A, reflecting data from lines of pixels. As shown in FIG. 4, image sensor waveform 283A may include analog data from lines of pixels. For example, FIG. 4 illustrates two pixel lines. The number of lines of pixels shown in FIG. 4 is merely for ease of illustration, and any suitable lines/numbers of pixels may be utilized. Image sensor waveform 283A during time period P₁ may reflect one frame of image sensor 282A (or an alternative number of frames). During second period P₂, image sensor 282A may be between frames, and thus image sensor waveform 283A may not include (i.e., lack) pixel data from image sensor 282A. During third period P₃, image sensor 282A may again be transmitting pixel data (e.g., in a second frame). During period P₄, image sensor 282A may once again be between frames and thus not transmitting pixel data in image sensor waveform 283A.

Referring to exemplary image sensor waveform 283A of FIG. 4, the downwards square waves, or syncs, between each pixel line in first period P₁ and third period P₃ are illustrative of exemplary output signals from analog image sensors. FPGA 196 may be configured to detect the downwards square waves between each pixel line, for example, to keep track of when active pixels begin in the waveform. Additionally or alternatively, FPGA 296 may be configured to detect the downwards square waves to stay in sync with the pixel data being received from the image sensor. Although downwards square raves are referenced, it will be appreciated that waveform 283A may include one or more of any suitable type of indicator. The one or more indicators may include, for example, a downwards square wave or any other indicator that FPGA 196 is configured to detect.

In some embodiments, FPGA 196 may be configured to detect the downwards square waves with a comparator, or an operational amplifier (“op amp”), that is triggered when the signal is below or above a voltage threshold. The comparator may monitor the signal and, when the signal goes above or below the voltage threshold, control switching mechanism 184 (e.g., to switch among sensors 82A, 82B, 82C, so as to select from which of sensors 182A, 182B, 182C to transmit the signal). Alternatively, the voltage threshold may be implemented in the digital domain in FPGA 196. In such a way, the voltage threshold may be implemented such that, when pixel data is not being transmitted (e.g., during second period P₂ or fourth period P₄ shown in FIG. 4), a signal from FPGA 196 is transmitted such that switch 189 of switching mechanism 184 is moved between sensors (e.g., between first sensor 182A, second sensor 182B, third sensor 182C, etc.). Accordingly, data may be received from additional sensors between frames. In some examples, a downwards square wave or other indicator between frames (e.g., during second period P₂ or fourth period P₄) may be provided to indicate to FPGA 196 that active data is about to begin.

The time between frames (i.e., a length of second period P₂ and fourth period P₄) may be determined by the configuration of image sensor 282A. For example, image sensor 282A may be configured such that there is a fixed time between each frame. The approximate time between each frame may vary depending on one or more of the number of pixels, framerate, pixel clock, and other aspects specific to the image sensor.

While the pixel data is inactive from image sensor 282A, for example, between frames during periods P₂ and P₄, switching mechanism 284 may switch to pressure sensor 282B. For example, switching mechanism 284 may switch from image sensor 282A to pressure sensor 282B at times t₁ (at an end of P₁/start of P₂) and t₃ (at an end of P₃/start of P₄). Although two sensors (e.g., image sensor 282A and pressure sensor 282B) are depicted in FIG. 4, it will be appreciated that switching mechanism 284 may be configured to switch among additional sensors between the frames or at other times. For example, switching mechanism 284 may be configured to first switch to pressure sensor 282B for a set timeframe (e.g., the time between each frame) during P₂/P₄ and then to a third sensor for a set timeframe (e.g., the time between each frame), still during P₂/P₄. Once the next frame of the pixel data from image sensor 282A begins (e.g., at t₂ or at t₄), switching mechanism 284 may switch back to the image sensor 282A.

In some examples, FPGA 196 may be configured to detect the downwards square waves or other indicators (for example, between frames), such that FPGA 296 stays in sync with the pixel data being received from the image sensor. For example, once FPGA 196 detects a downward square wave during second period P₂, FPGA 196 may be configured to send instructions to switching mechanism 184. Once the switching mechanism receives instructions from FPGA 196, the switching mechanism 184 may switch back to receiving pixel data from image sensor 282A.

Composite waveform 285 may thus be created by switching between each sensor 282A, 282B. For example, during time period P₁ (when switching mechanism 284 is switched to image sensor 282A), a first portion 287A of composite waveform 285 may include data from image sensor 282A. First portion 287A of composite waveform 285 may be the waveform generated by the first frame of image sensor 282A. A second portion 287B of composite waveform 285 may be a waveform generated by pressure sensor 282B during time period P₂. Additional portions of the composite waveform may be generated in similar manners, from image sensor 282A, pressure sensor 282B, and/or additional or alterative sensors. As described with respect to FIG. 3, the composite waveform is transmitted via wire 191 to analog processing unit 192, ADC 194, FPGA 196, and then output 198.

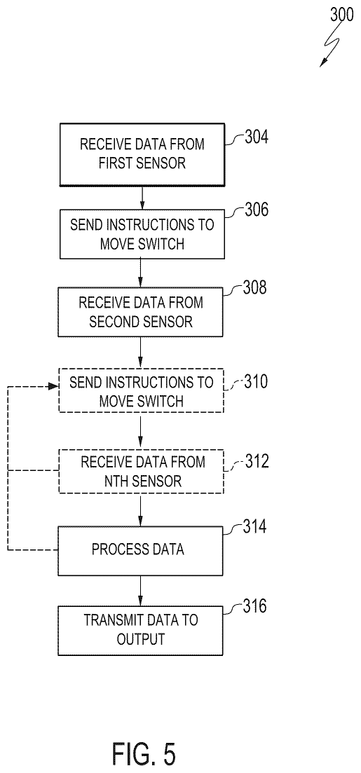

As discussed above, with respect to FIG. 3, FPGA 196 may be configured to control an operation of switching mechanism 284. In some examples, FPGA 196 may be configured to switch among image sensor 282A, pressure sensor 282B, and/or other sensors, at predetermined times (e.g., t₁, t₂, t₃, t₄, etc.). FPGA 196 or another component may store data regarding times at which switching mechanism 284 is operated to switch among sensors. FPGA 196 may utilize the stored data regarding the times at which switching mechanism 284 is operated in order to analyze composite waveform 285 to interpret data from the various sensors (e.g., image sensor 282A and pressure sensor 282B). FIG. 5 is a flow diagram of an exemplary method 300 that one or more aspects of medical system 10 of FIG. 1 or medical system 100 described above with respect to FIG. 3 may perform. Although references below are to aspects of medical system 100, it will be appreciated that elements of medical system 10 may also perform such steps. In an example, steps of method 300 may be performed by one or more elements of processing unit 165, such as, for example, FPGA 196.

First step 304 includes receiving data from first sensor 182A, for example, via wire 191 of FIG. 3. During step 304, switching mechanism 184 may be connected to first sensor 182A. As previously discussed, the transmitted data from first sensor 182A may be received by FPGA 196 after processing by analog processing unit 192 and/or ADC 194.

In a next step 306, processing unit 165 (e.g., FPGA 196) may transmit instructions (e.g., a first instruction) to move switching mechanism 184 to a second configuration. In the second configuration, switching mechanism 184 is connected to second sensor 182B. As discussed above, step 306 may be performed using wire 197.

Once switching mechanism 184 is connected to second sensor 182B, in a step 308, data from second sensor 182B may be received, e.g., via wire 191. As discussed above, with respect to step 304, the transmitted data from second sensor 182B may be received by FPGA 196 after processing by analog processing unit 192 and/or ADC 194.

Steps 310 and 312 may optionally repeat steps 306 and 308 for an Nth sensor 312N. It will be appreciated that sensor 312N may include sensors 312A and 312B, for which data was received in steps 304 and 308. Steps 310 and 312 may be performed as many times as desired in order to receive data from a desired number and pattern of sensor(s).

The data from first sensor 182A and second sensor 182B (and any other sensors) received in steps 304, 308, and, optionally, 310 may form a composite waveform, such as composite waveform 285, as illustrated in FIG. 4, and discussed above. Composite waveform 285 is created containing data from first sensor 182A followed by data from second sensor 182B. For example, first portion 287A of waveform 285 may correspond to data received in step 304. Second portion 287B of waveform 285 may correspond to data received in step 308. Data from additional sensors may follow data from second sensor 182B.

In step 316, FPGA 196 or another element of processing unit 165 may process data received in steps 304, 308, and, optionally 310. For example, as discussed above, such processing may include reconstructing the separate waveforms for each sensor 312A, 312B, and optionally, 312N. For example, FPGA 196 or a memory associated therewith may store information regarding how switching mechanism 184 was modulated in steps 306, 310, and, optionally, step 314. After step 314, further data may be received by optionally repeating steps 312, 314.

In a next step 316, the data from first sensor 182A, second sensor 182B, etc. may be sent to an output, such as output 198. As previously described, output 198 may include a display, a monitor, or a memory. Following step 316, any of the previous steps may be repeated one or more times, for example, throughout a procedure.

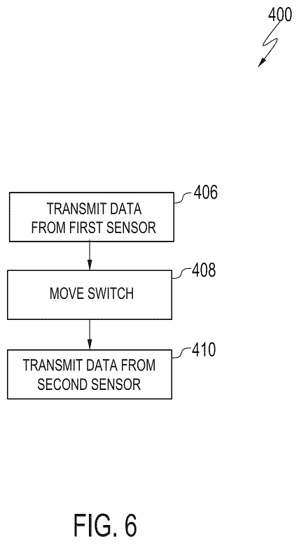

FIG. 6 is a flow diagram of an alternative exemplary method 400 that medical device 15 (of medical system 10 of FIG. 1) or medical device 115 (of medical system 100 described above with respect to FIG. 3) may perform. Although aspects of medical system 100 are referenced below, it will be appreciated that aspects of medical system 10 may be used additionally or alternatively. As previously discussed, medical system 100 may include, for example, medical device 115 and processing unit 165.

First step 406 includes transmitting data from first sensor 182A disposed on a distal portion of medical device 15 or 115. As discussed above with respect to FIG. 3, data may be transmitted from first sensor 182A when switching mechanism 184 is connected to first sensor 182A. Data may be transmitted via wire 191, through medical device 115.

In a next step 408, switch 189 on switching mechanism 184 may move to connect to second sensor 182B. With switch 189 connected to second sensor 182B, data is no longer transmitted from first sensor 182A.

In a next step 410, data is transmitted from second sensor 182B. Data may be transmitted via wire 191, through medical device 115.

Steps 406-410 may be repeated one or more times, for example, throughout a procedure. Additionally, method 400 may include additional steps, for example, according to the number of sensors disposed on medical device 15 or 115. For example, in a configuration with three sensors, method 400 may include a first additional step to move sensor from second sensor 182B to third sensor 182C and a second additional step to transmit data from third sensor 182C. In such a way, additional sensors may require additional steps.

In some examples, ADC 194 (illustrated in FIG. 3) may be configured to perform oversampling (e.g., the analog signal from the image sensor is sampled and/or converted at a much higher rate). For example, a sampling rate of ADC 194 may be set to be higher than the pixel rate of image sensor 282A. With this configuration, because additional samples for each pixel value are being acquired, switching mechanism 184 may be modulated to switch between sensors (e.g., between image sensor 282A and pressure sensor 282B) when the pixel data (e.g., from image sensor 282A) is still active. The resulting composite waveform may include the pixel data and data from other sensor samples.

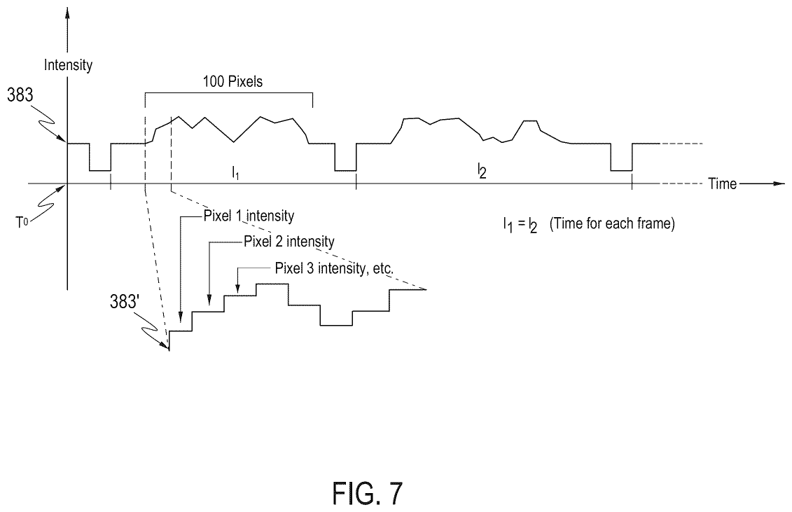

FIG. 7 illustrates an alternative exemplary analog signal/waveform 383, Waveform 383 may be similar to waveform 283A of FIG. 4, for example, during time period P₁ and/or time period P₂. Waveform 383 may be generated by an exemplary sensor, such as, for example, image sensor 282A. As previously described, alternative types of sensors may form waveform 383. The horizontal axis of the graph on which waveform 383 is illustrated represents time. The time scale may be nanoseconds, microseconds, milliseconds, seconds, or any other time interval to appropriately display data from image sensor 282A. For ease of illustration, two intervals of time, I₁ and I₂, are delineated by vertical ticks on the horizontal axis. Each time interval, I₁ and I₂, may be the same amount of time. In some examples, waveform 383 may include additional time intervals.

The vertical axis on which waveform 383 is illustrated represents an intensity of the pixels (although the vertical axis may be representative of other values for example, if other sensors are utilized). The intensity values of the pixels may be representative of corresponding voltage outputs from the sensors. In some examples, each interval of time, I₁ and I₂, may be representative of approximately 100 pixel intensity values. In other examples, each interval may be representative of fewer or additional pixel intensity values.

Still referring to FIG. 7, waveform 383′ is a magnified portion of waveform 383. Waveform 383′ illustrates details of a portion of waveform 383. Specifically, waveform 383′ illustrates exemplary output data for several pixels. For ease of illustration, three pixel intensity lines are labeled on waveform 383′ (Pixel 1 intensity, Pixel 2 intensity, Pixel 3 intensity). Additional pixel intensity lines are shown in the magnified portion of waveform 383. Each pixel (e.g., pixel 1, pixel 2, pixel 3, etc.) may generate several data points over time, thus generating horizontal lines (pixel 1 intensity, pixel 2 intensity, pixel 3 intensity) for each pixel. The horizontal lines may be generated in a series, thus generating waveform 383′, and, ultimately, waveform 383. For example, a series of values may be received from the first pixel for approximately 0.001 seconds (1×10⁶ ns). Next, a series of values may be received from the second pixel for approximately 0.001 seconds. Additional values may be received from additional pixels in sequence. Over time, the values of intensity from waveform 383 may be used to ultimately generate a live digital image.

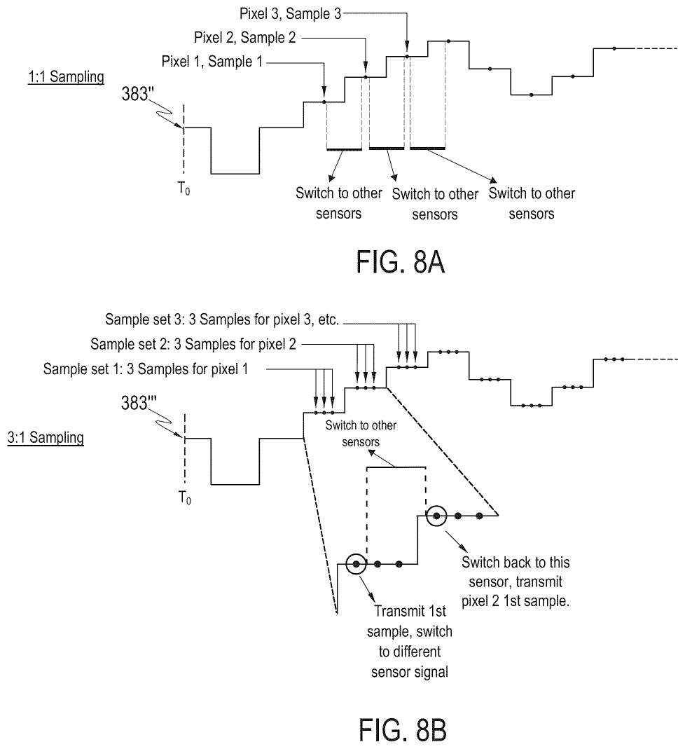

FIGS. 8A and 8B illustrate different sampling schemes of waveform 383′. For example, FIG. 8A illustrates an exemplary 1:1 sampling scheme of a waveform 383″. In this sampling scheme, ADC 194 (illustrated in FIG. 3) may be configured to perform sampling on a 1:1 scale. For example, ADC 194 may be configured to take a single data point, or sample, from each pixel (e.g., pixel 1, sample 1; pixel 2, sample 2; pixel 3, sample 3). Each data point, or sample, (e.g., pixel 1, sample 1; pixel 2, sample 2; pixel 3, sample 3, etc.), may be transmitted to FPGA 196 (illustrated in FIG. 3) to ultimately generate a live image that may be transmitted to and/or displayed on output 198. The samples may be taken at discrete or constant intervals or randomly.

Once FPGA 196 receives a sample from ADC 194 (e.g., pixel 1, sample 1), FPGA 196 may be configured to transmit an electrical signal through wire 197 to change switch 189 from terminal to terminal (e.g., from first terminal 188A to second terminal 188B, from second terminal 188B to third terminal 188C, etc.), as previously described. Because switch 189 moves between terminals, (e.g., first terminal 188A, second terminal 188B, third terminal 188C, etc.) and, thus, between sensors (e.g., sensors 182A, 182B, 182C . . . 182N), a composite waveform from the sensors is produced (e.g., as shown in FIG. 4) and transmitted via wire 191. With this configuration, switching mechanism 184 may be modulated to switch between sensors during active pixel data. For example, instead of switching between frames of the image sensor as discussed above with respect to FIG. 4, switching mechanism 184 is configured to switch between sensors (e.g., sensors 182A, 182B, 182C . . . 182N) between the pixel samples (e.g., between pixel 1, sample 1; pixel 2, sample 2; pixel 3, sample 3).

Although not shown, the composite waveform of this configuration may include sensor samples interspersed with the pixel data. For example, the composite waveform may include data in the following order: pixel 1, sample 1 data; data from one or more of the other sensors (e.g., sensors 182A, 182B, 182C . . . 182N); pixel 2, sample 2 data; data from one or more of the other sensors; pixel 3, sample 3 data; data from one or more of the other sensors, etc.

This composite waveform may undergo similar processing methods to the ones discussed above. For example, this composite waveform may then be transmitted via wire 191 to analog processing unit 192, where the waveform may be filtered, amplified, and/or level shifted, as discussed above. In such a way, signal-to-noise ratio may be improved. The modified composite waveform may then be transmitted to ADC 194, where the waveform may be converted from the analog waveform to a digital signal. The digital signal may then be transmitted from ADC 194 to FPGA 196. The digital signal may be stored or sorted by FPGA 196, for example, to reconstruct the separate waveforms for each sensor. For example, FPGA 196 or a memory associated therewith may store information regarding how switch 189 was modulated (e.g., at which time and to which sensor 182A, 182B, 182C . . . 182N switch 189 was modulated between each pixel).

Alternatively, FPGA 196 or another component of processing unit 165 may analyze characteristics of the signal to identify known characteristics of signals from the sensor(s) 182A, 182B, 182C . . . 182N. FPGA 196 may be electrically coupled to an output (e.g., a monitor, a screen, a second processor, etc.) 198, either directly or indirectly (e.g., via other processing components). Information from FPGA 196 regarding first sensor 182A, second sensor 182B, third sensor 182C, etc. may then be displayed on output 198, stored, or undergo additional processing.

FIG. 8B illustrates an alternative sampling scheme of a waveform 383″. In this sampling scheme, ADC 194 (illustrated in FIG. 3) may be configured to perform sampling on a 3:1 scale. This sampling scheme may be an oversampling technique. For example, ADC 194 may be configured to perform sampling at a higher sampling rate than what is otherwise necessary to acquire or process a signal. For example, ADC 194 may be configured to take three data points, or samples, from each pixel (e.g., 3 samples from pixel 1; 3 samples from pixel 2; 3 samples from pixel 3, etc.) from each pixel at discrete intervals. The three data points, or samples, from each pixel may form a sample set (e.g., sample set 1 from pixel 1 samples; sample set 2 from pixel 2 samples; sample set 3 from pixel 3 samples, etc.)

Although a 3:1 sampling scheme is shown, alternative sampling schemes may be considered. For example, ADC 194 may be configured to take two data points, or samples, from each pixel (a 2:1 sampling scheme), four data points from each pixel (a 4:1 sampling scheme), etc.

In the 3:1 sampling scheme, ADC 194 may be configured to save and transmit just one of the three samples from sample set (e.g., one sample from sample set 1 of pixel 1; one sample from sample set 2 of pixel 2; one sample from sample set 3 of pixel 3, etc.). For example, ADC 194 may be configured to transmit the first sample from each pixel. In alternative examples, ADC 194 may be configured to transmit the second sample from each pixel, or the third sample from each pixel, etc. In further alternative configurations, ADC 194 may be configured to transmit the first sample from sample set 1 of pixel 1, the second sample from sample set 2 of pixel 2, the third sample from sample set 3 of pixel 3, etc. The samples from each pixel may ultimately generate a live image that may be transmitted to and/or displayed on output 198.

In further alternative examples, ADC 194 may be configured to transmit the first sample and the third sample each sample set (e.g., the first and third sample from sample set 1 of pixel 1, the first and third sample from sample set 2 of pixel 2, the first and third sample from sample set 3 of pixel 3, etc.). For example, in this configuration, ADC 194 may be configured to transmit multiple samples from each pixel.

Similar to the sampling scheme described above, once FPGA 196 receives a sample from ADC 194 (e.g., first sample from sample set 1 of pixel 1), FPGA 196 may be configured to transmit an electrical signal through wire 197 to change switch 189 from terminal to terminal (e.g., from first terminal 188A to second terminal 188B, from second terminal 188B to third terminal 188C, etc.), as previously described. Because switch 189 moves between terminals, (e.g., first terminal 188A, second terminal 188B, third terminal 188C, etc.) and, thus, between sensors (e.g., sensors 182A, 182B, 182C . . . 182N), a composite waveform from the sensors is produced (shown in FIG. 4) and transmitted via wire 191.

With this configuration, switching mechanism 184 may be modulated to switch between sensors when the pixel data is still active. For example, instead of switching between frames of the image sensor, as discussed above with respect to FIG. 4, switching mechanism 184 may be configured to switch between sensors (e.g., sensors 182A, 182B, 182C . . . 182N) between the pixel samples.

Although not shown, the composite waveform of this configuration may include sensor samples interspersed with the pixel data. For example, the composite waveform may include, data in the following order: first sample from sample set 1 of pixel 1; data from one or more of the other sensors (e.g., sensors 182A, 182B, 182C . . . 182N); first sample from sample set 2 of pixel 2; data from one or more of the other sensors; first sample from sample set 3 of pixel 3 data; data from one or more of the other sensors, etc.

This composite waveform may undergo similar processing methods to the ones discussed above. For example, this composite waveform may then be transmitted via wire 191 to analog processing unit 192, where the waveform may be filtered, amplified, and/or level shifted, as discussed above. In such a way, noise may be reduced from the composite waveform. The modified composite waveform may then be transmitted to ADC 194, where the waveform may be converted from the analog waveform to a digital signal. The digital signal may then be transmitted from ADC 194 to FPGA 196. The digital signal may be stored or sorted by FPGA 196, for example, to reconstruct the separate waveforms for each sensor. For example, FPGA 196 or a memory associated therewith may store information regarding how switch 189 was modulated (e.g., at which time and to which sensor 182A, 182B, 182C . . . 182N switch 189 was modulated between each pixel).

Alternatively, FPGA 196 or another component of processing unit 165 may analyze characteristics of the signal to identify known characteristics of signals from the sensor(s) 182A, 182B, 182C . . . 182N. FPGA 196 may be electrically coupled to an output (e.g., a monitor, a screen, a second processor, etc.) 198, either directly or indirectly (e.g., via other processing components). Information from FPGA 196 regarding first sensor 182A, second sensor 182B, third sensor 182C, etc. may then be displayed on output 198, stored, or undergo additional processing.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

We claim:

1. A medical system, comprising:
a processing unit,
a medical device electrically coupled to the processing unit, wherein the medical device includes an insertion portion configured to be inserted into a body lumen of a subject; and
an electronic assembly disposed at a distal portion of the insertion portion, wherein the electronic assembly includes at least a first sensor, a second sensor, and a switch, wherein the electronic assembly is electrically coupled to the processing unit, and wherein the switch is configured to switch between the first sensor and the second sensor based on an instruction of the processing unit.

2. The medical system of claim 1, wherein the medical device further includes a conducting element configured to transmit a composite waveform comprising signals from the first sensor and the second sensor.

3. The medical system of claim 2, wherein the composite waveform is transmitted via a single conducting element.

4. The medical system of claim 2, wherein the conducting element extends through a shaft of the medical device.

5. The medical system of claim 1, wherein the processing unit includes a field programmable gate array (“FPGA”), wherein the FPGA is configured to generate the instruction to switch the switch between the first sensor and the second sensor.

6. The medical system of claim 5, wherein the instruction to switch between the first sensor and the second sensor is transmitted from the FPGA to the electronic assembly via a cable electrically coupled to the FPGA and the electronic assembly.

7. The medical system of claim 5, wherein the medical system further includes an output, wherein the output is electrically coupled to the FPGA, wherein the FPGA is configured to transmit data received from a composite waveform to the output.

8. The medical system of claim 1, wherein the switch is electrically coupled to an analog processing unit.

9. The medical system of claim 8, wherein the analog processing unit is electrically coupled to an analog to digital converter.

10. The medical system of claim 9, wherein the analog to digital converter is electrically coupled to an FPGA.

11. The medical system of claim 1, wherein the electronic assembly includes a third sensor, and wherein the switch is configured to switch between the first sensor, the second sensor, and the third sensor based on the instruction received from the processing unit.

12. The medical system of claim 1, wherein each of the first sensor and the second sensor is an analog sensor.

13. The medical system of claim 1, wherein the first sensor and the second sensor are each one of an image sensor, a temperature sensor, a humidity sensor, a light sensor, a flow rate sensor, a pressure sensor, an oximeter, a glucometer, a heart rate sensor, a respiration rate sensor, a force sensor, an airflow sensor, a position and/or orientation sensor, a magnetic field sensor, and a pH sensor.

14. The medical system of claim 1, wherein the electronic assembly includes a first portion and a second portion, wherein the first portion includes the first sensor and the switch, and wherein the second portion includes the second sensor.

15. The medical system of claim 14, wherein the first portion is disposed on a first surface of the medical device, and wherein the second portion is disposed on a second surface of the medical device, wherein the first surface is angled relative to the first surface.

16. A medical method comprising:
receiving data from a first sensor when a switch is in a first configuration;
sending a first instruction to move the switch from the first configuration to a second configuration;
receiving data from a second sensor when the switch is in the second configuration; and
transmitting the data received from the first sensor and the second sensor to a processing unit.

17. The medical method of claim 16, further comprising:
analyzing the transmitted data from the first sensor and the second sensor in order to reconstruct data from the first sensor and the second sensor.

18. The medical method of claim 17, wherein the transmitted data is analyzed based on stored data of the first instruction to move the switch.

19. The medical method of claim 16, further comprising:
sending a second instruction to move the switch from the second configuration to a third configuration, wherein, in the third configuration, data is received from a third sensor.

20. An electronic assembly of a medical device, the electronic assembly comprising:
a first sensor;
a second sensor;
a switch having a primary terminal and configured to switch between a first terminal electrically coupled to the first sensor and a second terminal electrically coupled to the second sensor; and
a conductive element electrically coupled to the primary terminal, wherein the conductive element is configured to transmit a composite waveform comprising signals from the first sensor and the second sensor.
